(19) United States
(12) Patent Application Publication
(10) Pub. No.: US 2025/0020621 A1
(43) Pub. Date: Jan. 16, 2025

(54) POLE-MOUNTED DEVICES AND SYSTEMS FOR IOT-ENABLED MONITORING OF METHANE EMISSIONS OF ONE OR MORE INDUSTRIAL FACILITIES

(71) Applicant: Schlumberger Technology Corporation, Sugar Land, TX (US)

(72) Inventors: Raphael Gadot, Sugar Land, TX (US); Vincent Nicolas, Missouri City, TX (US); Corey Ray, Richmond, TX (US)

(21) Appl. No.: 18/834,888

(22) PCT Filed: Apr. 28, 2023
(86) PCT No.: PCT/US2023/020386

§ 371 Date: Jul. 31, 2024

Related U.S. Application Data
(60) Provisional application No. 63/363,958, filed on May 2, 2022.

(51) Int. Cl. G01N 33/00; G06Q 30/018

(52) U.S. Cl. CPC G01N 33/0027; G01N 33/0075; G06Q 30/018

(57) ABSTRACT

The disclosure relates to an emissions detector for monitoring methane emissions at one or more industrial facilities. The emissions detector includes: a pole; an enclosure mounted on the pole, wherein the enclosure houses at least one sensor, wherein the at least one sensor includes a gas sensor configured to measure concentration of methane in atmospheric gas that flows into the enclosure; and means for removably securing the pole to ground without the use of concrete.

### CROSS-REFERENCE TO RELATED APPLICATIONS

The subject disclosure claims priority from U.S. Provisional Appl. No. 63/363,958, filed on May 2, 2022, herein incorporated by reference in its entirety.

### FIELD

The subject disclosure relates to systems and methods for monitoring methane emissions at industrial facilities, such as oil and gas facilities including well sites, compressor stations, and processing facilities.

### BACKGROUND

Methane emissions in the oil and gas industry are receiving intense scrutiny as it is believed that such methane emissions contribute to global warming and/or climate change. A large portion of the methane emissions in the oil and gas industry arise from a small number of major emission events henceforth referred to as super-emitters. Super-emitters occur from a variety of sites, and recent data suggest many emission events are intermittent.

Continuous monitoring using methane emissions detectors installed permanently at a site offers an effective way to identify, quantify, and mitigate intermittent methane emissions. However, installing methane emissions detectors across many diverse sites can be economically challenging.

### SUMMARY

In embodiments, an emissions detector for monitoring methane emissions at one or more industrial facilities is provided that includes a pole with an enclosure mounted on the pole. The enclosure houses at least one sensor, which includes a gas sensor configured to measure concentration of methane in atmospheric gas that flows into the enclosure. The emissions detector also includes means for removably securing the pole to ground without the use of concrete.

In embodiments, the means for removably securing the pole to ground can include a ground anchor with an exterior thread that removably screws into the ground. The ground anchor can have an interior hollow channel that receives and surrounds a bottom section of the pole.

In other embodiments, the means for removably securing the pole to the ground can include a tripod base and a plurality of ground screws that interface to the tripod base. The plurality of ground screws can each have an exterior thread that removably screw into the ground. The tripod base can have an interior hollow channel that receives and surrounds a bottom section of the pole.

In embodiments, the at least one sensor of the enclosure can further include at least one atmospheric sensor configured to measure properties of atmospheric gas that flows into the enclosure.

In embodiments, the emissions detector can further include acquisition and communication electronics mounted on the pole, wherein the acquisition and communication electronics are operably coupled to the enclosure by at least one cable.

In embodiments, the emissions detector can further include at least one solar panel mounted on the pole.

In embodiments, the emissions detector can further include an anemometer mounted on the pole.

In embodiments, the emissions detector can further include a camera or LIDAR device mounted on the pole and/or a gateway device mounted on the pole.

In embodiments, automated systems and methods are provided for methane emissions monitoring of one or more industrial facilities. The automated systems and methods can be economically deployed worldwide on a large scale. The automated systems and methods employ a network of emissions detectors for each industrial facility to be monitored. The emissions detectors of the network are spaced from one another at different locations at the industrial facility. The emissions detectors of the network are configured to perform time-series measurements of methane concentration at different locations within the industrial facility and communicate time-series sensor data representing such measurements (i.e., high-frequency location-specific methane concentration data) to an edge gateway device located at the industrial facility (or located within communication range from the industrial facility).

One or more emissions detectors of the network can also include one or more atmospheric sensors that are configured to perform time-series measurements of atmospheric conditions (such as temperature, atmospheric pressure, and humidity) at one or more locations within the industrial facility. The one or more emissions detectors with atmospheric sensor(s) are further configured to communicate times-series sensor data representing such measurements (i.e., high-frequency location-specific atmospheric data) to the edge gateway device.

One or more emissions detectors of the network can also include one or more environmental sensors that are configured to perform time-series measurements of environmental conditions (such as wind speed, wind direction, and solar radiation) at one or more locations within the industrial facility. The one or more emissions detectors with environmental sensor(s) are further configured to communicate times-series sensor data representing such measurements (i.e., high-frequency location-specific environmental data) to the edge gateway device.

Each emissions detector of the network can include a Global Navigation Satellite System (GNSS) module that precisely tracks the position of the emissions detector and time. The sensor data communicated from a given emissions detector of the network can be stamped with location and time information as recorded by the Global Navigation Satellite System (GNSS) module in synchronization with the time-series measurements performed by the given mission detector.

The edge gateway device can be configured to collect, aggregate, and process the time-series sensor data (i.e., the high-frequency location-specific methane concentration data and the high-frequency location-specific atmospheric data and the high-frequency location-specific environmental data) communicated from the network of emissions detectors, for example, by filtering and/or averaging the sensor data to derive corresponding time-series operational data and forwards such time-series operational data to a remote cloud computing environment. The time-series operational data represents i) methane concentration at specific locations within the industrial facility as a function of time as derived from the location-specific raw methane concentration data, ii) atmospheric conditions at specific location(s) within the one or more industrial facility as a function of time as derived from the location-specific atmospheric data, and iii) environmental conditions at specific location(s) within the one or more industrial facility as a function of time as derived from the location-specific environmental data. The remote cloud computing environment receives the time-series operational data communicated from the edge gateway device and processes the received time-series operational data using a suitable computational model, such as Gaussian plume dispersion model, to characterize methane emission at the industrial facility. For example, the cloud computing environment can process the received time-series operational data using a suitable computational model (e.g., Gaussian plume dispersion model) to detect the presence of methane emission at the industrial facility, the location of the methane emission at the industrial facility (when present), and the associated rate of methane emission at the industrial facility (when present). The cloud computing environment can generate data related to the methane emission (such as the location of the methane emission at the industrial facility, and the associated rate of methane emission at the industrial facility) and process such data to automatically generate an alert characterizing the methane emission at the industrial facility. The alert can be communicated to a designated party or user to initiate or schedule mitigation of the methane emission at the industrial facility.

In embodiments, each emissions detector of the network can include electronics that provide for wireless data communication between the emissions detector and the edge gateway device. In embodiments, the wireless data communication can implement a predefined wireless communication protocol, such as the LoRaWAN protocol. The LoRaWAN protocol employs spread spectrum modulation techniques derived from chirp spread spectrum (CSS) technology and provides a range up to 10 km with a data rate of up to 50 kbps.

In embodiments, the network of emissions detectors can be deployed over locations within one or more oil and gas facilities, such as one or more well sites, one or more compressor stations, or one or more processing facilities.

In embodiments, the edge gateway device can be configured to collect and process time-series sensor data measured by the emissions detector networks at multiple industrial facilities (e.g., multiple well sites), and the cloud computing environment can be configured to process time-series operational data derived from the time-series sensor data measured by the emissions detector networks at the multiple industrial facilities to characterize methane emission at the respective industrial facilities.

In other embodiments, the emissions detectors of the network(s) can be configured to process the real-time measurement data, for example, including filtering and/or averaging, and forward the resultant time-series data to the gateway device. The gateway device can collect, aggregate, and process such data for communication to the cloud computing environment.

In still other embodiments, the gateway device can be configured to process time-series operational data derived from the measurements performed by the network(s) of emissions detectors operably coupled thereto using a suitable computational model, such as Gaussian plume dispersion model, to characterize methane emission at one or more industrial facilities. For example, the gateway device can process the time-series operational data using a suitable computational model (e.g., simulation and inversion using a Gaussian plume dispersion model) to detect the presence of methane emission at one or more industrial facilities, the location of the methane emission at one or more industrial facilities (when present), and the associated rate of methane emission at the one or more industrial facilities (when present). The gateway device can be further configured to generate data related to the methane emission (such as the location of the methane emission at the one or more industrial facilities, and the associated rate of methane emission at the one or more industrial facilities) and process such data to automatically generate alert(s) characterizing the methane emission at the one or more industrial facilities. The alert(s) can be communicated to designated party (ies) or user(s) to initiate or schedule mitigation of the methane emission at the one or more industrial facilities.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIG. 1 is a schematic block diagram that depicts an automated system for methane emissions monitoring in accordance with an embodiment of the subject disclosure;

FIG. 2 is a schematic block diagram that depicts another automated system for methane emissions monitoring in accordance with an embodiment of the subject disclosure;

FIGS. 3A and 3B, collectively, is a flow chart of operations that can be carried out by the cloud computing environment of FIGS. 1 and 2;

FIG. 4 is a schematic diagram that depicts the automated system for methane emissions monitoring of FIG. 1 deployed at a well site (e.g., well pad);

FIG. 5 is a schematic diagram that depicts the automated system for methane emissions monitoring of FIG. 1 deployed at another well site (e.g., well pad);

FIG. 6 is a schematic diagram of a pole-mounted emissions detector with one or more environmental sensor(s) in accordance with the present disclosure, which is suitable for use as part of the system of FIG. 1;

FIG. 7 is a diagram of a pole-mounted emissions detector with one or more environmental sensor(s) in accordance with the present disclosure;

FIG. 8 is a diagram of another pole-mounted emissions detector with one or more environmental sensor(s) in accordance with the present disclosure;

FIG. 9 is a photo of the pole-mounted emissions detector with one or more environmental sensor(s) of FIG. 8; and

FIG. 10 is a schematic diagram of an example computing system.

### DETAILED DESCRIPTION

The particulars shown herein are by way of example and for purposes of illustrative discussion of the embodiments of the subject disclosure only and are presented in the cause of providing what is believed to be the most useful and readily understood description of the principles and conceptual aspects of the subject disclosure. In this regard, no attempt is made to show structural details in more detail than is necessary for the fundamental understanding of the subject disclosure, the description taken with the drawings making apparent to those skilled in the art how the several forms of the subject disclosure may be embodied in practice. Furthermore, like reference numbers and designations in the various drawings indicate like elements.

The present disclosure is directed to automated systems and methods that provide for methane emissions monitoring of one or more industrial facilities. The automated systems and methods can be economically deployed worldwide on a large scale.

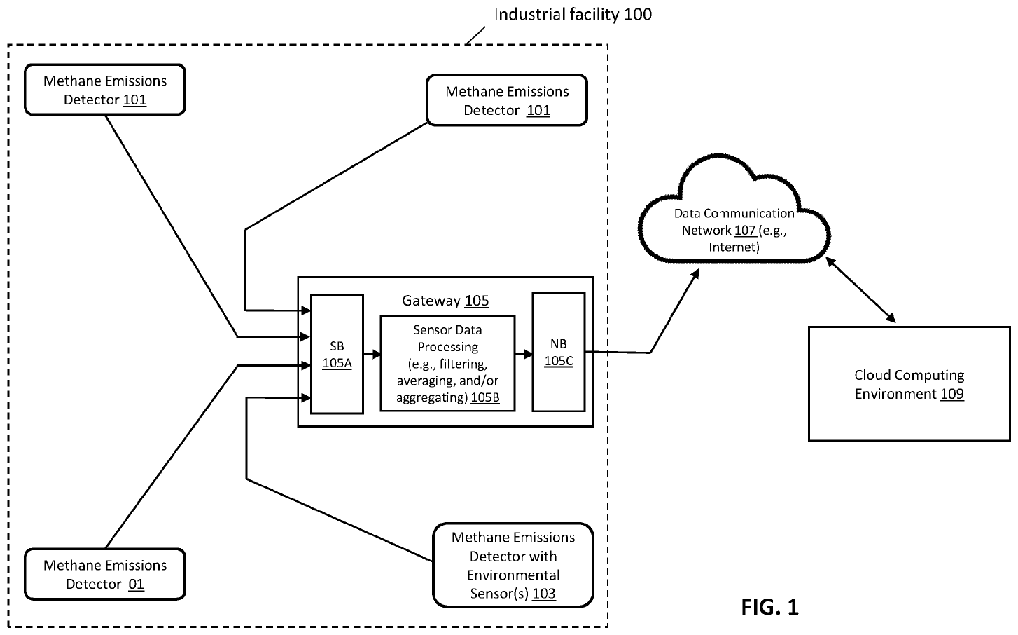

In an example embodiment shown in FIG. 1, an automated system is provided that includes an edge gateway device 105 that is located at or near (e.g., at a distance of 10 km or less) an industrial facility 100. The edge gateway device 105 is a ruggedized computing device that can be configured to deliver performance edge computing and secure data ingestion. The edge gateway device 105 can be configured to enable real-time monitoring and control of the operational equipment at the industrial facility 100.

The edge gateway device 105 can be configured to receive, collect, and aggregate data from a variety of operational equipment at the industrial facility 100 (such as sensors, controllers, actuators, programmable logic controllers, remote terminal units, and supervisory control and data acquisition (SCADA) systems), prepare such data for transmission to the remote cloud computing environment 109, and transmit the data from the edge gateway device 105 to the remote cloud computing environment 109 over a data communication network 107 as shown in FIG. 1. The data communication network 107 can include a cellular data network, satellite link, other Wide Area Network, the Internet, and/or another mode of available data communication. The cloud computing environment 109 can be implemented by one or more processor-based systems as is well known.

In embodiments, the edge gateway device 105 can employ a compact and rugged NEMA/IP rated housing for outdoor use, making it suitable for the environments at well sites and facilities. The overall packaging can also be environmentally qualified.

In embodiments, the gateway device 105 can be configured with a bi-directional communication interface (referred to as a Southbound Interface labeled SB, 105A) for data communication to the operational equipment at the facility 100 using either a wired communication protocol (such as a serial, Ethernet, Modbus or Open Platform Communication (OPC) protocol) or a wireless communication protocol (such as IEEE 802.11 Wi-Fi protocol, Highway Addressable Remote Transducer Protocol (HART), LoraWAN, WiFi or Message Queuing Telemetry Transport (MQTT)). The Southbound Interface 105A can provide for direct data communication to the operational equipment at the facility 100. Alternatively, the Southbound Interface 105A can provide for indirect data communication to the operational equipment at the facility 100 via a local area network or other local communication devices.

In embodiments, the edge gateway device 105 can be configured with a bi-directional communication interface (referred to as a Northbound Interface labeled NB, 105C) to the data communication network 107 using a wireless communication protocol. In embodiments, the wireless communication protocol can employ cellular data communication, such as 4G LTE data transmission capability (or possibly 3G data transmission for fallback capability). For facilities without a cellular signal, the Northbound Interface 105C to the data communication network 107 can be provided by a bidirectional satellite link (such as a BGAN modem). Alternatively, the Northbound Interface 105C can implement other wireless communication protocols or one or more wired communication protocols implemented by the data communication network 107.

In embodiments, the edge gateway device 105 can employ an embedded processing environment (e.g., data processor and memory system) that hosts and executes an operating system and application(s) or module(s) as described herein.

In embodiments, the edge gateway device 105 can employ both hardware-based and software-based security measures. The hardware-based security measures can involve a hardware root-of-trust established using an industry-standard Trusted Platform Module (TPM) v2.0 cryptographic chip. The software-based security measures can include operating system hardening and encryption of both buffered and transmitted data.

In embodiments, the edge gateway device 105 can support a containerized microservice-based architecture. This architecture enables extensibility into several distinct and different solutions for different environments and applications at the edge, while still using the same infrastructure components. In embodiments, the edge gateway device 105 can employ one or more containers to implement one or more applications or modules executing on the gateway device 105 that perform functionality for methane emissions monitoring as described herein. A container is a standard unit of software that packages up code and all its dependencies (such as runtime environment, system tools, system libraries and settings) so that the application or module runs quickly and reliably in the computing environment of the edge gateway device 105. The container isolates the software from its environment and ensures that it works uniformly and reliably in the computing environment of the edge gateway device 105.

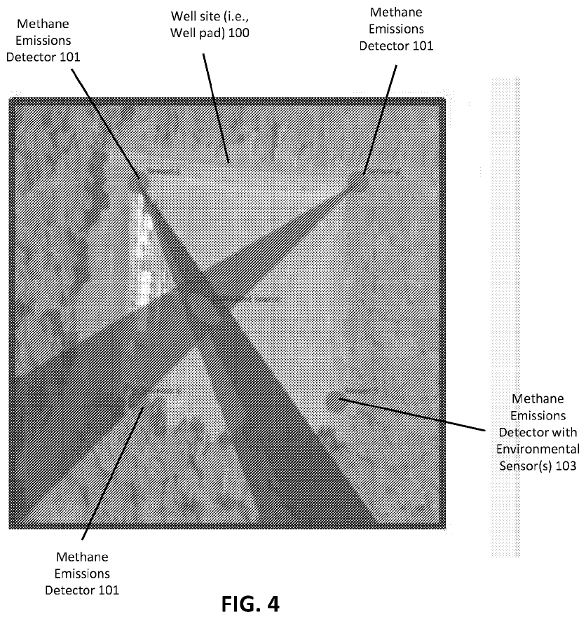

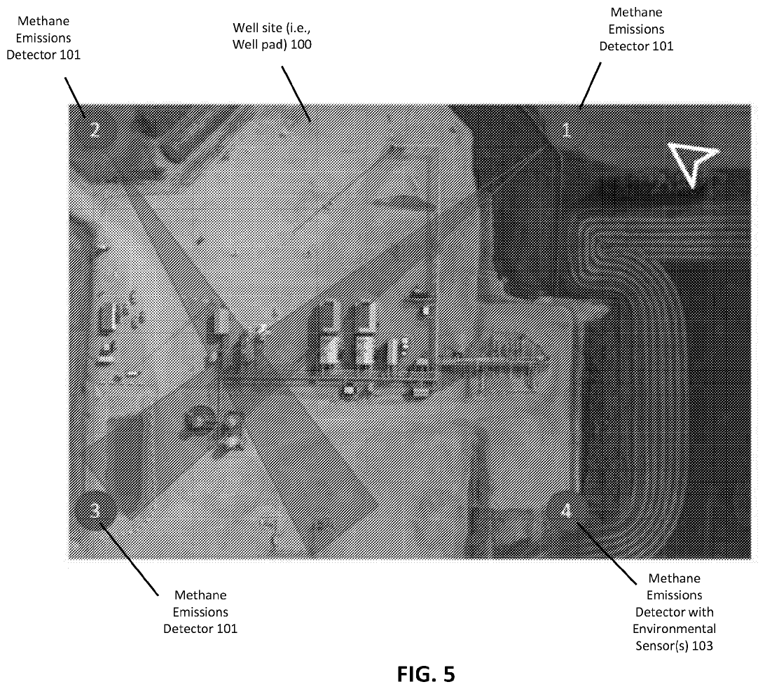

In embodiments, the Southbound Interface 105A of the gateway device 105 utilizes a wireless communication protocol to interface to a network of methane emissions detectors (for example, three methane emissions detectors 101 and one methane emissions detector with environmental sensor(s) 103) that are spaced from one another at different locations at the industrial facility 100, for example, in opposed corners of the facility as shown in FIGS. 4 and 5. The network of methane emissions detectors 101, 103 is configured to perform time-series measurements of methane concentration at different locations within the industrial facility 100 and communicate time-series sensor data representing such measurements (i.e., high-frequency location-specific methane concentration data) to the edge gateway device 105.

In embodiments, one or more methane emissions detectors of the network can include one or more atmospheric sensors that are configured to perform time-series measurements of atmospheric conditions (such as temperature, atmospheric pressure, and humidity) at one or more locations within the industrial facility 100. The one or more methane emissions detectors with atmospheric sensor(s) can be further configured to communicate time-series sensor data representing such measurements (i.e., high-frequency location-specific atmospheric data) to the edge gateway device 105. In embodiments, the atmospheric sensor(s) can be selected from the group that includes: one or more sensors that measure temperature and humidity, and a barometer for measuring atmospheric pressure.

In embodiments, one or more methane emissions detectors of the network, such as the methane emissions detector 103 in FIG. 1, can include one or more environmental sensors that are configured to perform time-series measurements of environmental conditions (such as wind speed, wind direction, and solar radiation) at one or more locations within the industrial facility 100. The one or more methane emissions detectors with environmental sensor(s) can be further configured to communicate time-series sensor data representing such measurements (i.e., high-frequency location-specific environmental data) to the edge gateway device 105. In embodiments, the environmental sensors can be selected from the group that includes: an anemometer for measuring wind speed and wind direction, and a pyranometer for measuring solar radiation.

In embodiments, each methane emissions detector (101, 103) of the network can include a Global Navigation Satellite System (GNSS) module that precisely tracks the position of the methane emissions detector and time. The time-series sensor data communicated from a given methane emissions detector (101, 103) can be stamped with location and time information as recorded by the Global Navigation Satellite System (GNSS) module in synchronization with the time-series measurements performed by the given methane emissions detector (101, 103).

In embodiments, each methane emissions detector (101, 103) of the network can include a gas sensor for measuring methane concentration in the immediate vicinity of the gas sensor together with the electronics that interface to the gas sensor and provide for data communication between the methane emissions detector (101, 103) and the edge gateway device 105. In embodiments, the data communication can implement a predefined wireless communication protocol, such as the LoRaWAN protocol. The LoRaWAN protocol employs spread spectrum modulation techniques derived from chirp spread spectrum (CSS) technology and provides a range up to 10 km with a data rate of up to 50 kbps.

The edge gateway device 105 includes sensor data processing functionality 105B that is configured to collect, aggregate, and process the time-series sensor data (i.e., the high-frequency location-specific raw methane concentration data and the high-frequency location-specific raw environmental data) communicated from the network of methane emissions detectors 101, 103, for example, by filtering and/or averaging the data, to generate corresponding time-series operational data. The time-series operational data represents i) methane concentration at specific locations within the industrial facility 100 as a function of time as derived from the location-specific methane concentration data, ii) atmospheric conditions at specific location(s) within the industrial facility 100 as a function of time as derived from the location-specific atmospheric data, and iii) environmental conditions at specific location(s) within the industrial facility 100 as a function of time as derived from the location-specific environmental data. The sensor data processing functionality 105B is further configured to cooperate with the Northbound Interface 105C to forward the time-series operational data to the remote cloud computing environment 109 via the data communication network 107. The cloud computing environment 109 receives the time-series operational data communicated from the edge gateway device 105 and processes the received time-series operational data to detect the presence of methane emission at the industrial facility 100, the location of the methane emission at the industrial facility 100 (when present), and the associated rate of methane emission at the industrial facility 100 (when present). The cloud computing environment 109 can generate data related to the methane emission (such as the location of the methane emission at the industrial facility 100, and the associated rate of methane emission at the industrial facility 100) and process such data to automatically generate an alert characterizing the methane emission. The alert can be communicated to a designated party or user to initiate or schedule mitigation of the methane emission at the industrial facility 100.

In embodiments, the cloud computing environment 109 can employ a suitable computational model, such as Gaussian plume dispersion model, to detect the presence of methane emission at the industrial facility 100, the location of the methane emission at the industrial facility 100, and the associated rate of methane emission at the industrial facility 100. In this embodiment, the computational model can be used as part of simulation and inversion operations that detects the presence of methane emission at the industrial facility 100, the location of the methane emission at the industrial facility 100, and the associated rate of methane emission at the industrial facility 100.

In embodiments, each methane emissions detector (101, 103) of the network can include an accelerometer for measuring the acceleration of the methane emissions detector. Detector-specific raw acceleration data can be communicated from the methane emissions detectors (101, 103) of the network to the edge gateway device 105 and processed by the edge gateway device 105 to detect fall events for the respective methane emissions detectors 101, 103. Additionally or alternatively, the edge gateway device 105 can derive detector-specific acceleration data from such measurements, and communicate the detector-specific acceleration data from the edge gateway device 105 to the cloud computing environment 109. The cloud computing environment 109 can be configured to receive and process the detector-specific acceleration data to detect fall events for the respective methane emissions detectors (101, 103) of the network. The alert can be communicated to a designated party or user to initiate or schedule repair of the fallen emissions detector at the industrial facility 100.

In embodiments, the edge gateway device 105 can be configured to collect and process time-series sensor data measured by methane emissions detector networks at multiple industrial facilities (e.g., multiple well sites), and the cloud computing environment 109 can be configured to process time-series operational data derived from the time-series sensor data measured by the methane emissions detector networks at the multiple industrial facilities to characterize methane emission at the respective industrial facilities.

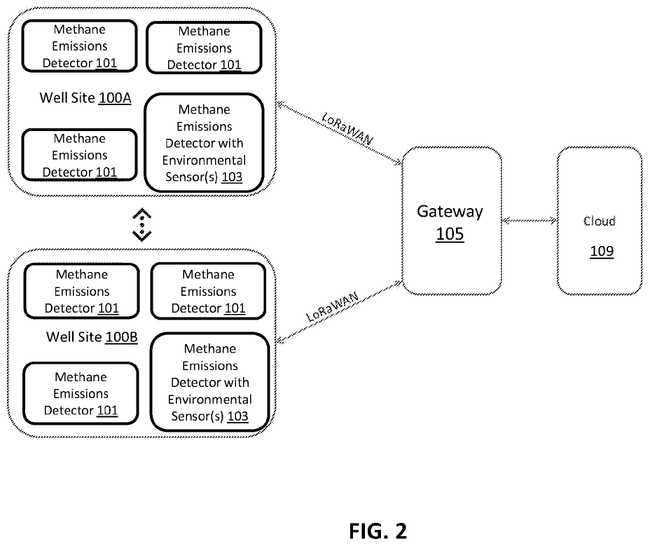

For example, in another example embodiment shown in FIG. 2, the edge gateway device 105 (e.g., the Southbound Interface 105A) utilizes a wireless communication protocol to interface to multiple networks of methane emissions detectors for monitoring emissions for two well sites 100A and 100B as shown in FIG. 2. In this embodiment, the methane emissions detectors of each network are spaced from one another at different locations within the respective well sites 100A, 100B. For example, the network of methane emissions detectors (101, 103) for the well site 100A can be deployed in opposed corners of the well site 100A as shown in FIG. 4, while the network of methane emissions detectors (101, 103) for the well site 100B can be deployed in opposed corners of another well site 100B as shown in FIG. 5. The multiple networks of methane emissions detectors (101, 103) are configured to perform time-series measurements of methane concentration at different locations within the corresponding well sites 100A, 100B and wirelessly communicate time-series sensor data representing such measurements (i.e., high-frequency location-specific methane concentration data) to the edge gateway device 105.

In embodiments, one or more methane emissions detectors of each network can include one or more atmospheric sensors that are configured to perform time-series measurements of atmospheric conditions (such as temperature, atmospheric pressure, and humidity) at one or more locations within the respective well sites 100A, 100B. The one or more methane emissions detectors with atmospheric sensor(s) can be further configured to wirelessly communicate time-series sensor data representing such measurements of atmospheric conditions (i.e., high-frequency location-specific atmospheric data) to the edge gateway device 105. In embodiments, the atmospheric sensors can be selected from the group that includes: one or more sensors that measure temperature and humidity, and a barometer for measuring atmospheric pressure. Furthermore, one or more methane emissions detectors of each network, such as the methane emissions detector 103 for well site 100A and the methane emissions detector 103 for well site 100B in FIG. 2, can include one or more environmental sensors that are configured to perform time-series measurements of environmental conditions (such as wind speed, wind direction, and solar radiation) at one or more locations within the respective well sites 100A, 100B. The one or more methane emissions detectors with environmental sensor(s), such as the methane emissions detector 103 for well site 100A and the methane emissions detector 103 for well site 100B, can be further configured to wirelessly communicate time-series sensor data representing such measurements of environmental conditions (i.e., high-frequency location-specific environmental data) to the edge gateway device 105. In embodiments, the environmental sensors can be selected from the group that includes: an anemometer for measuring wind speed and wind direction, and a pyranometer for measuring solar radiation).

In embodiments, each methane emissions detector (101, 103) of the multiple networks can include a Global Navigation Satellite System (GNSS) module that precisely tracks the position of the methane emissions detector and time. The time-series sensor data representing the time-series measurements communicated from a given methane emissions detector (101, 103) can be stamped with location and time information as recorded by the Global Navigation Satellite System (GNSS) module in synchronization with the time-series measurements performed by the given methane emissions detector (101, 103).

In embodiments, each methane emissions detector (101, 103) of the multiple networks can include a gas sensor for measuring methane concentration in the immediate vicinity of the gas sensor together with the electronics that interface to the gas sensor and provide for data communication between the methane emissions detector (101, 103) and the edge gateway device 105. In embodiments, the data communication can implement a predefined wireless communication protocol, such as the LoRaWAN protocol as shown in FIG. 2.

The edge gateway device 105 includes sensor data processing functionality 105B that is configured to collect, aggregate, and process the time-series data (i.e., the high-frequency location-specific methane concentration data and the high-frequency location-specific atmospheric data and the high-frequency location-specific environmental data) communicated from the multiple networks of methane emissions detectors 101, 103, for example, by filtering and/or averaging the data, to generate time-series operational data. The time-series operational data represents i) methane concentration at specific locations within the well sites 100A and 100B as a function of time as derived from the location-specific methane concentration data from the well sites 100A and 100B, ii) atmospheric conditions at specific location(s) within the well sites 100A and the 100B as a function of time as derived from the location-specific atmospheric data from well sites 100A and 100B, and iii) environmental conditions at specific location(s) within the well sites 100A and 100B as a function of time as derived from the location-specific environmental data from well sites 100A and 100B. The sensor data processing functionality 105B is further configured to cooperate with the Northbound Interface 105C to forward such time-series operational data to the remote cloud computing environment 109 via the data communication network 107. The cloud computing environment 109 receives the time-series operational data communicated from the edge gateway device 105 and processes the received time-series operational data for each well site to detect the presence of methane emission at the respective well sites 100A, 100B, the location of the methane emission at the respective well sites 100A, 100B (when present), and the associated rate of methane emission at the respective well sites 100A, 100B (when present). The cloud computing environment 109 can generate data related to the methane emission for the respective well sites 100A, 100B (such as the location of the methane emission for the respective well sites 100A, 100B, and the associated rate of methane emission for the respective well sites 100A, 100B) and process such data to automatically generate an alert characterizing methane emission for the respective well sites 100A, 100B. The alert can be communicated to a designated party or user to initiate or schedule mitigation of the methane emission at the respective well sites 100A, 100B.

In embodiments, the cloud computing environment 109 can employ suitable computational models, such as Gaussian plume dispersion models, to detect the presence of methane emission at the respective well sites 100A, 100B, the location of the methane emission at the respective well sites 100A, 100B (when present), and the associated rate of methane emission at the respective well sites 100A, 100B (when present). In this case, the computational models for the respective well sites 100A, 100B can be used as part of separate simulation and inversion operations that detect the presence of methane emission at the respective well sites 100A, 100B, the location of the methane emission at the respective well sites 100A, 100B (when present), and the associated rate of methane emission at the respective well sites 100A, 100B (when present).

In this manner, the edge gateway device 105 can be configured to collect and process time-series sensor data measured by methane emissions detector networks at multiple industrial facilities (e.g., multiple well sites), and the cloud computing environment can be configured to process operational data derived from the time-series sensor data measured by the methane emissions detector networks at the multiple industrial facilities to detect the presence of methane emission at the respective industrial facilities, the location of the methane emission at the respective industrial facilities (when present), and the associated rate of methane emission at the respective industrial facilities (when present).

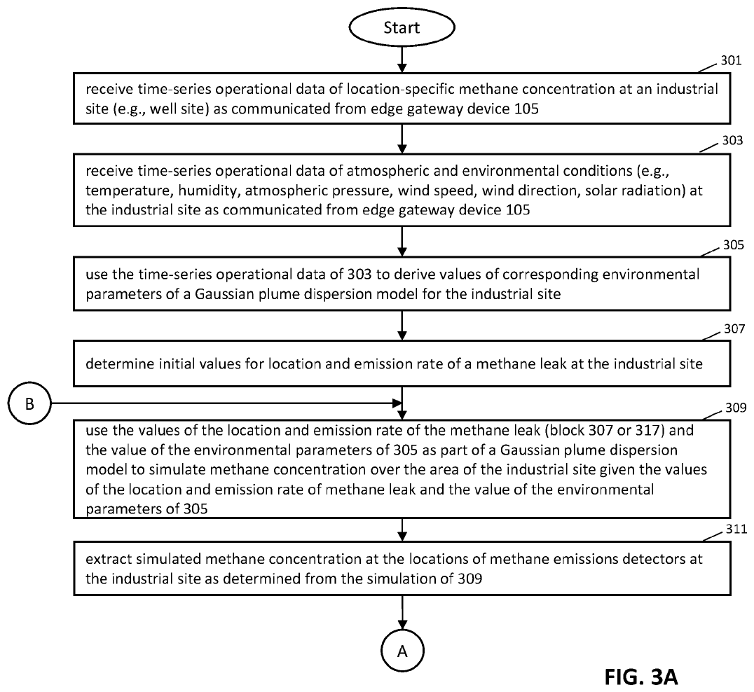

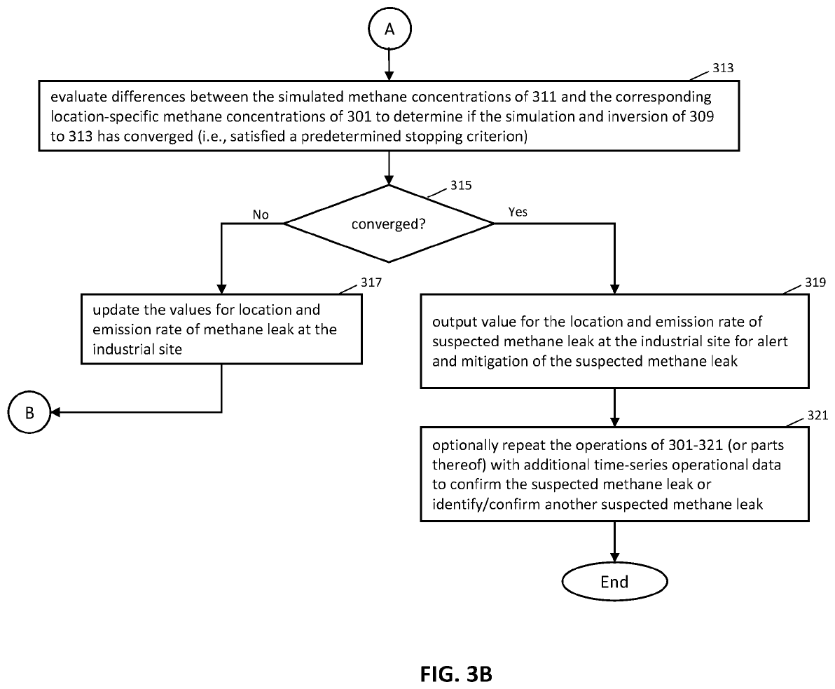

FIGS. 3A and 3B, collectively, is a flow chart of operations that can be carried out by the cloud computing environment 109 of FIGS. 1 and 2.

The operations begin in block 301 where the cloud computing environment 109 is configured to receive time-series operational data of location-specific methane concentration at an industrial site (e.g., a well site) as communicated from edge gateway device 105.

In block 303, the cloud computing environment 109 is configured to receive time-series operational data of location-specific atmospheric and environmental conditions (e.g., temperature, atmospheric pressure, humidity, wind speed, wind direction, solar radiation) at the industrial site as communicated from the edge gateway device 105.

In block 305, the cloud computing environment 109 is configured to use the time-series operational data of 303 to derive values of corresponding environmental parameters of a Gaussian plume dispersion model for the industrial site.

In block 307, the cloud computing environment 109 is configured to determine initial values for the location and emission rate of a methane leak at the industrial site.

In block 309, the cloud computing environment 109 is configured to use the values for the location and emission rate of the methane leak (block 307 or 317) and the value of the environmental parameters of 305 as part of a Gaussian plume dispersion model for the industrial site. The cloud computing environment 109 runs (or executes) the Gaussian plume dispersion model to simulate methane concentration over the area of the industrial site given the values for the location and emission rate of methane leak and the value of the environmental parameters of 305.

In block 311, the cloud computing environment 109 is configured to extract simulated methane concentration at the locations of methane emissions detectors at the industrial site as determined from the simulation of 309.

In block 313, the cloud computing environment 109 is configured to evaluate differences between the simulated methane concentrations of 311 and the corresponding location-specific methane concentrations of 301 to determine if the simulation and inversion of 309 to 313 has converged (i.e., satisfied a predetermined stopping criterion).

In block 315, the cloud computing environment 109 determines if the simulation and inversion of 309 to 313 has converged. If not, the operations continue to block 317. If so, the operations continue to blocks 319 to 321.

In block 317, the cloud computing environment 109 is configured to update the values for the location and emission rate of methane leak at the industrial site, and the processing returns to block 309.

In block 319, the cloud computing environment 109 is configured to output values for the location and emission rate of suspected methane leak at the industrial site for alert and mitigation of the suspected methane leak. In embodiments, the last values for the location and emission rate of methane leak that resulted in convergence as determined in block 315 can be output as the values for the location and the emission rate of suspected methane leak at the industrial site.

In block 321, the cloud computing environment 109 can be configured to optionally repeat the operations of 301-321 (or parts thereof) with additional time-series operational data to confirm the suspected methane leak or identify/confirm another suspected methane leak.

In other embodiments, the methane emissions detectors of the network(s) can be configured to process the real-time measurement data, for example, including filtering and/or averaging, and forward the resultant time-series data to the gateway device. The gateway device can collect, aggregate, and process such data for communication to the cloud computing environment.

In still other embodiments, the gateway device 105 can be configured to process time-series operational data derived from the measurements performed by the network(s) of methane emissions detectors operably coupled thereto using a suitable computational model, such as Gaussian plume dispersion model, to characterize methane emission at one or more industrial facilities. For example, the gateway device can process the time-series operational data using a suitable computational model (e.g., simulation and inversion using a Gaussian plume dispersion model) to detect the presence of methane emission at one or more industrial facilities, the location of the methane emission at one or more industrial facilities (when present), and the associated rate of methane emission at the one or more industrial facilities (when present). The gateway device can be further configured to generate data related to the methane emission (such as the location of the methane emission at the one or more industrial facilities, and the associated rate of methane emission at the one or more industrial facilities) and process such data to automatically generate alert(s) characterizing the methane emission at the one or more industrial facilities. The alert(s) can be communicated to designated party (ies) or user(s) to initiate or schedule mitigation of the methane emission at the one or more industrial facilities.

FIG. 4 depicts the automated system for methane emissions monitoring of FIG. 1 deployed at a well site (e.g., well pad).

FIG. 5 depicts the automated system for methane emissions monitoring of FIG. 1 deployed at another well site (e.g., well pad).

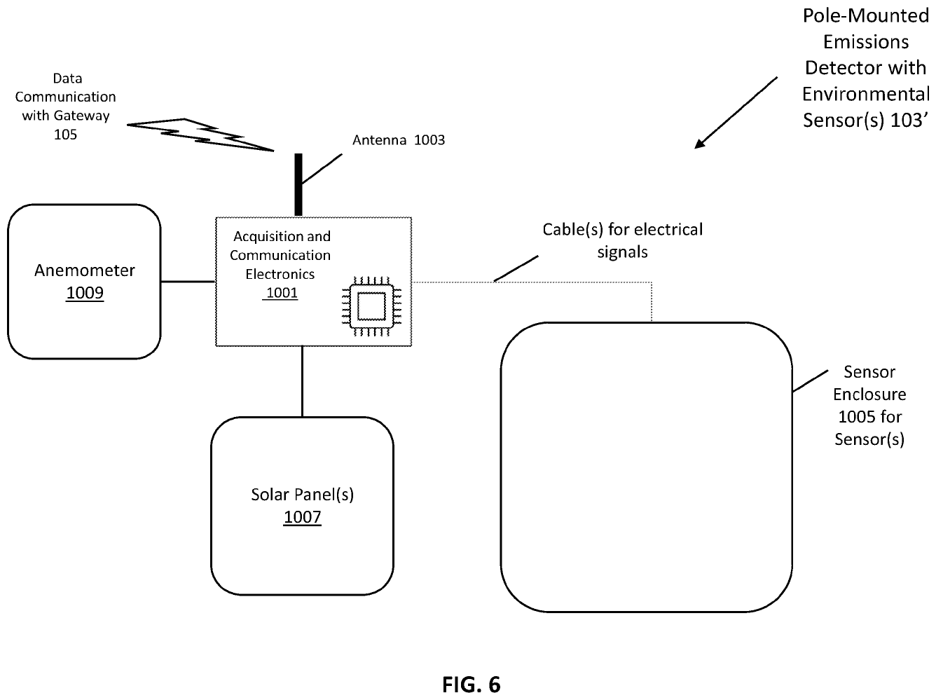

FIG. 6 is a schematic diagram of a pole-mounted emissions detector with one or more environmental sensor(s) 103′ in accordance with the present disclosure, which is suitable for use as part of the system of FIG. 1. The emissions detector 103′ includes acquisition and communication electronics 1001 and a sensor enclosure 1005 that are both mounted to a pole (not shown) for positioning above the ground. The acquisition and communication electronics 1001 includes electrical circuitry that interfaces to electrical interface components of the sensor enclosure 1005 via one or more cable(s) as shown. The acquisition and communication electronics 1001 further include additional electrical circuitry that supports data communication with the gateway device 105. In embodiments, the data communication can employ one or more RF antenna 1003 for wireless data communication according to one or more predefined wireless communication protocols (such as LoraWan, Zigbee, WiFi) as supported by the Southbound data communication interface of the gateway device 105. In other embodiments, the data communication can employ wired data communication, for example, via copper cables or fiber optic cables, which can be used for Ethernet or other wired data communication protocols with the gateway device 105. The electrical circuitry of the acquisition and communication electronics 1001 can be embodied by one or more printed circuit boards (PCBs) as is well known in the electronic arts. The acquisition and communication electronics 1001 can further include or interface to an electrical power source that supplies electrical power to the electrical components of the emissions detector 103′. In embodiments, the electrical power source can include one or more pole-mounted solar panels 1007 as shown, one or more batteries (e.g., lithium-ion battery (ies)), a wind-powered generator, mains power, and/or other suitable electrical power source.

The sensor enclosure 1005 mechanically supports a gas sensor and possibly at least one atmospheric sensor. The sensor enclosure 1005 is configured to permit atmospheric gas to flow into the enclosure and into space at or near the gas sensor, and the gas sensor can be configured to measure the concentration of methane in the atmospheric gas that flows into the enclosure and into the space at or near the gas sensor. The methane can be part of the atmospheric gas that flows into the space at or near the gas sensor due to a methane leak in the local vicinity of the sensor enclosure 1005. The sensor enclosure 1005 can also be configured to permit atmospheric gas to flow into the enclosure and into space at or near one or more atmospheric sensor(s), and the atmospheric sensor(s) can be configured to measure gas properties (such as temperature, atmospheric pressure, and humidity) of the atmospheric gas that flows into the enclosure and into the space at or near the atmospheric sensor(s). The gas sensor can output analog signals or digital data that represents the methane concentration measured by the gas sensor. Such analog signals or digital data can be supplied to electrical components of the sensor enclosure 1005 for processing and/or output to the acquisition and communication electronics 1001. The atmospheric sensor(s) can output analog signals or digital data that represents the atmospheric properties measured by the atmospheric sensor(s). Such analog signals or digital data can be supplied to electrical components of the enclosure 1005 for processing and/or output to the acquisition and communication electronics 1001.

The acquisition and communication electronics 1001 can further interface to a pole-mounted anemometer 1009, which can be configured to measured wind speed and wind direction in the local vicinity of the detector 103′.

In embodiments, the electrical components of the enclosure 1005 can provide for one or more of sampling, analog-to-digital (A-to-D) conversion, data filtering and averaging, and/or data communication with the acquisition and communication electronics 1001 via the cable(s) therebetween. Similarly, the electrical components of the acquisition and communication electronics 1001 can provide for one or more of sampling, A-to-D conversion, data filtering and averaging, and/or data communication with the electrical components of the enclosure 1005 via the cable(s) therebetween.

The acquisition and communication electronics 1001 can be configured to generate data representing the measurements made by the sensor(s) of the enclosure 1005 and the anemometer 1009 over time and communicate such operational data to the gateway device 105 for processing as described herein. Such data can represent raw measurements of the sensor(s) of the enclosure 1005 and the anemometer 1009 over time. Alternatively or additionally, such data can be derived from filtering and/or averaging the measurements of the sensor(s) of the enclosure 1005 and the anemometer 1009 over time. Similarly, the gateway device 105 can be configured to filter and/or average data that represents the raw measurements of the sensor(s) of the enclosure 1005 and the anemometer 1009 over time for processing as described herein.

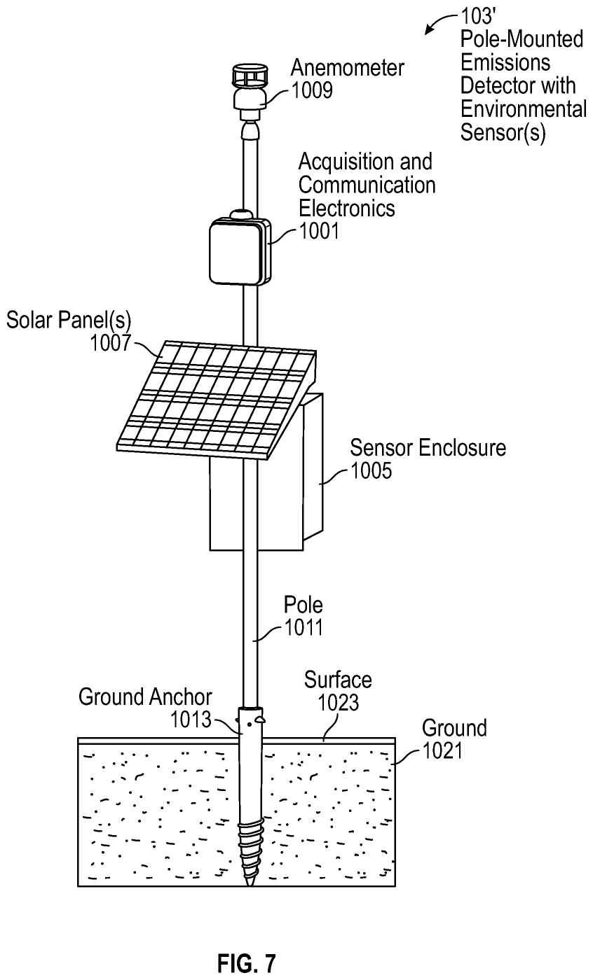

FIG. 7 is a diagram of a pole-mounted emissions detector with one or more environmental sensor(s) 103′ in accordance with the present disclosure. In this embodiment, the components of emissions detector 103′ of FIG. 6, including the acquisition and communication electronics 1001, the sensor enclosure with sensor(s) 1005, solar panel(s) 1007 and anemometer 1009, are mounted to a pole 1011. In embodiments, the pole 1011 is approximately two (2) inches in diameter. The pole 1011 can vary in height. For example, the height of the pole 1011 can be up to eleven (11) feet or more. The bottom section of the pole 1011 is secured to a ground anchor 1013 having an exterior thread that screws into the ground 1021 as shown. The ground anchor 1013 can be particularly adapted for use with soft soil (such as clay). In this configuration, the bottom section of the pole 1011 is received and surrounded by the interior hollow channel of the ground anchor 1013, and the ground anchor 1013 is screwed into the ground to secure the pole 1011 in a position that extends above the surface 1023 of the ground 1021 as shown. In order to remove the pole 1011 (with the system components if mounted thereon) and the ground anchor 1013 from the installation site, the pole 1011 can be separated from the ground anchor 1013 and the ground anchor 1013 can be unscrewed and removed from the ground without leaving any residual footprint in the ground once removed. This is advantageous over other installation methods that pour concrete to hold and support the pole in place.

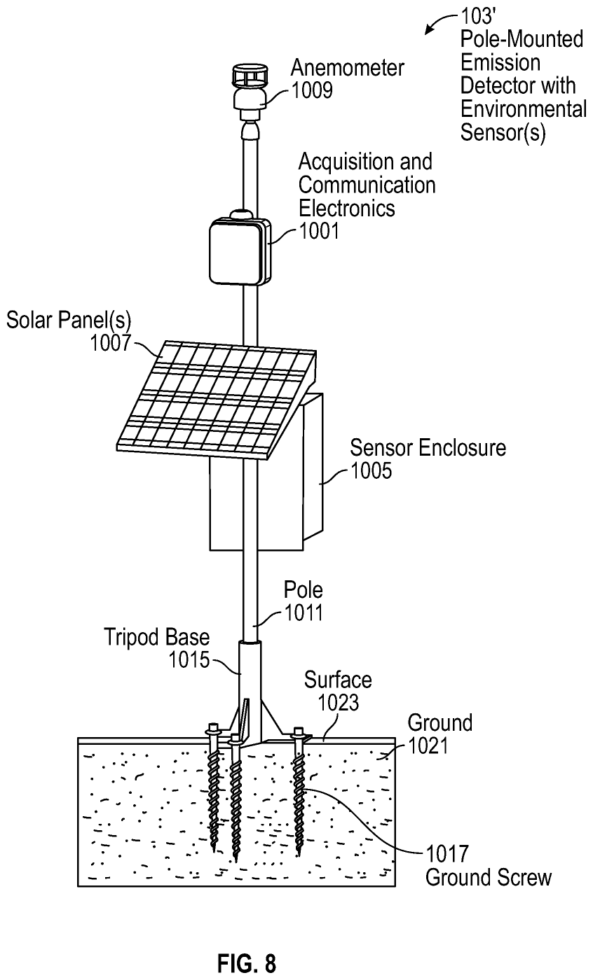

FIG. 8 is a diagram of another pole-mounted emissions detector with one or more environmental sensor(s) 103′ in accordance with the present disclosure. In this embodiment, the components of emissions detector 103′ of FIG. 6, including the acquisition and communication electronics 1001, the sensor enclosure with sensor(s) 1005, solar panel(s) 1007 and anemometer 1009, are mounted to a pole 1011. In embodiments, the pole 1011 is approximately two (2) inches in diameter. The pole 1011 can vary in height. For example, the height of the pole 1011 can be up to eleven (11) feet or more. The bottom section of the pole 1011 is secured to a tripod base 1015 that is secured to the surface 1023 of the ground 1021 by a number of ground screws 1017 (for example, three ground screws shown) that each have an exterior thread that screws into the ground 1021 as shown. The tripod base 1015 and the ground screws 1017 can be particularly adapted for use with hard soil (such as compacted gravel). In this configuration, the bottom section of the pole 1011 is received and surrounded by the interior hollow channel of the tripod base 1015, and the ground screws 1017 are screwed into the ground to secure the tripod base 1015 and the pole 1011 in a position where the pole 1011 extends above the surface 1023 of the ground 1021 as shown. In order to remove the pole 1011 (with the detector components if mounted thereon) and the tripod base 1015 and the ground screws 1017 from the installation site, the ground screws 1017 can be unscrewed and removed from the ground without leaving any residual footprint in the ground once removed. This is advantageous over other installation methods that pour concrete to hold and support the pole in place.

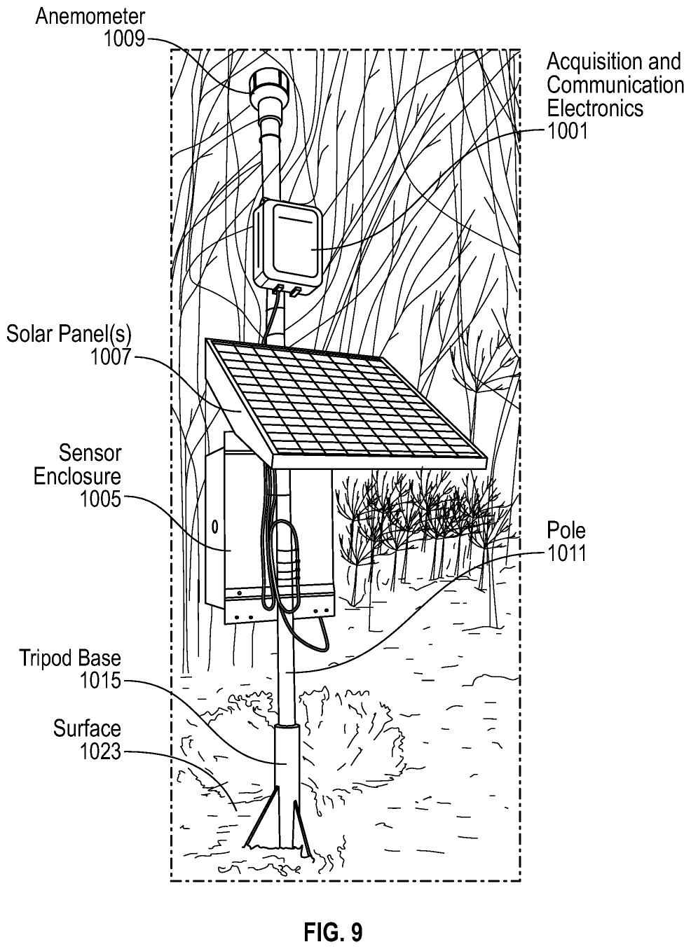

FIG. 9 is a photo of the pole-mounted emissions detector with one or more environmental sensor(s) 103′ of FIG. 8 deployed at a facility, such as a well pad.

Advantageously, the pole-mounted emissions detector of the present disclosure can be installed without excavating the ground and then using concrete to anchor the pole. This means that no cement is left in the ground when the detector is decommissioned. This is an environmentally friendly solution to deploy the pole-mounted emissions detector of the present disclosure at a facility, such as a well pad.

In addition, the pole-mounted emissions detector of the present disclosure can have a number of advantages, including: (a) semi-permanent installation; (b) easily removable; (c) portable (no heavy weight bags of cement to carry); (d) environmentally friendly as when decommissioning the facility nothing is left behind, no cement is left in the ground, no chemicals, etc.; (e) sustainable since ground can be reused once removed from a facility; (f) fast to install (no cement curing process involved); (g) wide environmental temperature range during installation, in contrast to cement which will not cure at low temperature; (h) no need to bring water to prepare cement; (i) no need to bring tools for cement preparation (wheelbarrow, cement mixer, tubs, etc.); and (j) no excavation required (which can require heavy machinery).

Furthermore, the pole-mounted emissions detector of the present disclosure is not limited to only installing a methane gas sensor on a pole, but it can also be applied to installing other sensors such as atmospheric sensors (atmospheric conditions: temperature, humidity, wind speed, wind direction, solar radiation, rain, lightning detection, etc.) on a pole, and cameras (RGB or infrared) or lidars on a pole. It can also be used to install communication devices such as wireless gateways, or edge computing devices independently from the sensors.

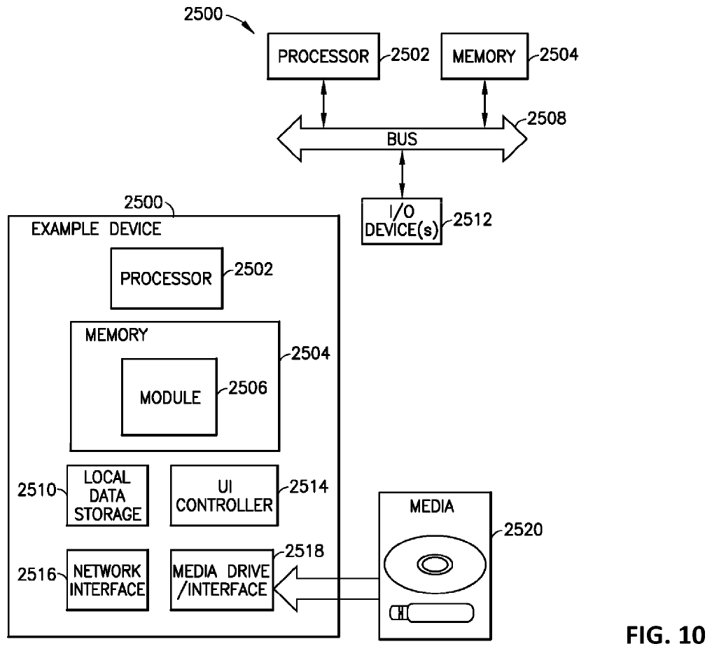

In some embodiments, the methods and system of the present disclosure may involve a computing system. FIG. 10 illustrates an example computing system 2500, with a processor 2502 and memory 2504 that can be configured to implement various embodiments of the automated systems and methods for methane emissions monitoring as discussed in the present application. Memory 2504 can also host one or more databases and can include one or more forms of volatile data storage media such as random-access memory (RAM), and/or one or more forms of nonvolatile storage media (such as read-only memory (ROM), flash memory, and so forth).

Device 2500 is one example of a computing device or programmable device and is not intended to suggest any limitation as to scope of use or functionality of device 2500 and/or its possible architectures. For example, device 2500 can comprise one or more computing devices, programmable logic controllers (PLCs), etc.

Further, device 2500 should not be interpreted as having any dependency relating to one or a combination of components illustrated in device 2500. For example, device 2500 may include one or more of computers, such as a laptop computer, a desktop computer, a mainframe computer, etc., or any combination or accumulation thereof.

Device 2500 can also include a bus 2508 configured to allow various components and devices, such as processors 2502, memory 2504, and local data storage 2510, among other components, to communicate with each other.

Bus 2508 can include one or more of any of several types of bus structures, including a memory bus or memory controller, a peripheral bus, an accelerated graphics port, and a processor or local bus using any of a variety of bus architectures. Bus 2508 can also include wired and/or wireless buses.

Local data storage 2510 can include fixed media (e.g., RAM, ROM, a fixed hard drive, etc.) as well as removable media (e.g., a flash memory drive, a removable hard drive, optical disks, magnetic disks, and so forth). One or more input/output (I/O) device(s) 2512 may also communicate via a user interface (UI) controller 2514, which may connect with I/O device(s) 2512 either directly or through bus 2508.

In one possible implementation, a network interface 2516 may communicate outside of device 2500 via a connected network. A media drive/interface 2518 can accept removable tangible media 2520, such as flash drives, optical disks, removable hard drives, software products, etc. In one possible implementation, logic, computing instructions, and/or software programs comprising elements of module 2506 may reside on removable media 2520 readable by media drive/interface 2518.

In one possible embodiment, input/output device(s) 2512 can allow a user (such as a human annotator) to enter commands and information to device 2500, and also allow information to be presented to the user and/or other components or devices. Examples of input device(s) 2512 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

Various systems and processes of present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable, and non-removable tangible media implemented for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information, and which can be accessed by a computer.

Some of the methods and processes described above, can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, general-purpose computer, special-purpose machine, virtual machine, software container, or appliance) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

Some of the methods and processes described above, can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

1. An emissions detector for monitoring methane emissions at one or more industrial facilities, the emissions detector comprising:
a pole;
an enclosure mounted on the pole, wherein the enclosure houses at least one sensor, wherein the at least one sensor includes a gas sensor configured to measure concentration of methane in atmospheric gas that flows into the enclosure; and
means for removably securing the pole to ground without the use of concrete.

2. The emissions detector of claim 1, wherein:
the means for removably securing the pole to ground comprises a ground anchor with an exterior thread that removably screws into the ground.

3. The emissions detector of claim 2, wherein:
the ground anchor has an interior hollow channel that receives and surrounds a bottom section of the pole.

4. The emissions detector of claim 1, wherein:
the means for removably securing the pole to the ground comprises a tripod base and a plurality of ground screws that interface to the tripod base, wherein the plurality of ground screws each have an exterior thread that removably screw into the ground.

5. The emissions detector of claim 4, wherein:
the tripod base has an interior hollow channel that receives and surrounds a bottom section of the pole.

6. The emissions detector of claim 1, wherein:
the at least one sensor of the enclosure further includes at least one atmospheric sensor configured to measure properties of atmospheric gas that flows into the enclosure.

7. The emissions detector of claim 1, further comprising:
acquisition and communication electronics mounted on the pole, wherein the acquisition and communication electronics are operably coupled to the enclosure by at least one cable.

8. The emissions detector of claim 1, further comprising:
at least one solar panel mounted on the pole.

9. The emissions detector of claim 1, further comprising:
an anemometer mounted on the pole.

10. The emissions detector of claim 1, further comprising:
a camera or LIDAR device mounted on the pole; and
a gateway device mounted on the pole.

11. A system for monitoring methane emissions at one or more industrial facilities, the system comprising:
a network of emissions detectors spaced from one another at different locations within an industrial facility;
a gateway device operably coupled to the network of emissions detectors; and
a cloud computing environment operably coupled to the gateway device;
wherein the network of emissions detectors is configured to perform time-series measurements at different locations within the industrial facility and wirelessly communicate time-series sensor data representing such measurements to the gateway device;
wherein the gateway device is configured to process the time-series sensor data to derive time-series operational data and communicate the time-series operational data to the cloud computing environment; and
wherein the cloud computing environment is configured to receive and process the time-series operational data to detect and characterize methane emission at the industrial facility.

12. The system of claim 11, wherein:
the cloud computing environment is configured to process the time-series operational data in conjunction with a computational model to determine a location of the methane emission at the industrial facility and an associated rate of methane emission at the industrial facility; and
the computational model comprises a Gaussian plume dispersion model.

13. (canceled)

14. The system of claim 11, wherein:
the time-series operational data represents methane concentration at specific locations within the industrial facility and environmental conditions at specific location(s) within the industrial facility as a function of time; and
wherein the cloud computing environment is configured to process such time-series operational data in conjunction with a computation model that simulates methane emission at the industrial facility based on environmental conditions within the industrial facility.

15. The system of claim 11, wherein:
the cloud computing environment is further configured to generate data related to the methane emission and process such data to automatically generate an alert characterizing the methane emission at the industrial facility.

16. The system of claim 11, wherein:
the gateway device is configured to collect and process time-series sensor data measured by emissions detector networks at multiple industrial facilities; and
the cloud computing environment is configured to process operational data derived from the time-series sensor data measured by the emissions detector networks at the multiple industrial facilities to characterize methane emission at the respective industrial facilities.

17. The system of claim 16, wherein:
the multiple industrial facilities comprises multiple oil and gas facilities such as one or more well sites, one or more compressor stations, or one or more processing facilities.

18. The system of claim 11, wherein:
the industrial facility comprises an oil and gas facility such as a well site, compressor station, or processing facility.

19. A system for monitoring methane emissions at one or more industrial facilities, the system comprising:
a network of emissions detectors spaced from one another at different locations within an industrial facility; and
a gateway device operably coupled to the network of emissions detectors;
wherein the network of emissions detectors is configured to perform time-series measurements at different locations within the industrial facility and wirelessly communicate time-series sensor data representing such measurements to the gateway device; and
wherein the gateway device is configured to process the time-series sensor data to derive time-series operational data and process the time-series operational data to detect and characterize methane emission at the industrial facility.

20. The system of claim 19, wherein:
the gateway device is configured to process the time-series operational data in conjunction with a computational model to determine a location of the methane emission at the industrial facility and an associated rate of methane emission at the industrial facility; and
the computational model comprises a Gaussian plume dispersion model.

21. (canceled)

22. The system of claim 19, wherein:
the gateway device is further configured to generate data related to the methane emission and process such data to automatically generate an alert characterizing the methane emission at the industrial facility.

23. (canceled)
